(19) United States
(12) Patent Application Publication
(10) Pub. No.: US 2006/0216361 A1
(43) Pub. Date: Sep. 28, 2006

(54) Compositions and methods for the treatment of osteoporosis and inflammatory joint disease

(76) Inventors: John B. Edwards, Atlanta, GA (US); Lori T. Erlandson, Atlanta, GA (US); E. Nicholas Griffin, Cumming, GA (US); Alan T. Roberts, Woodstock, GA (US); Jacob Selhub, Brookline, MA (US)

Correspondence Address: JOHN S. PRATT, ESQ;KILPATRICK STOCKTON, LLP, 1100 PEACHTREE STREET, ATLANTA, GA, 30309, US

(21) Appl. No.: 11/372,238

(22) Filed: Mar. 9, 2006

Related U.S. Application Data
(60) Provisional application No. 60/660,419, filed on Mar. 10, 2005.

(51) Int. Cl. A61K 33/06; A61K 31/525; A61K 31/714

(52) U.S. Cl. 424/682; 514/168; 514/251; 514/52; 514/350

(57) ABSTRACT

Compositions and methods for the treatment of osteoporosis and/or inflammatory joint disease are provided herein. The compositions contain a folate, such as a reduced folate, and folic acid. The folate is preferably 5-methyltetrahydrofolate, and most preferably 5-methyl-(6S)-tetrahydrofolic acid. The folate and folic acid can be given in the same dosage unit or separate dosage units, and more than one dosage unit can be given per dose. The compositions may also contain one or more vitamins and minerals selected from vitamin B12, vitamin B6, vitamin D3, calcium, magnesium, and polyunsaturated fatty acids (PUFAs). These ingredients are optional, but preferable (especially the vitamins and minerals). The compositions may further contain one or more additional ingredients such as vitamins, minerals, and laxatives. The compositions are useful in the treatment of all forms of osteoporosis, including primary osteoporosis and secondary osteoporosis, and/or inflammatory joint diseases, especially in patients having a folic acid metabolism deficiency. The compositions are particularly useful in the treatment of inflammatory joint diseases, with complications that include bone loss, fracture, and osteoporosis. In addition, the compositions are beneficial for the prevention of osteoporosis in subjects who do not yet have the disease, but who are at risk for getting osteoporosis, such as post-menopausal women, subjects with osteopenia (mid thinning of the bone mass), subjects with an inflammatory joint disease, or people who are over the age of 70.

### CROSS-REFERENCE TO RELATED APPLICATIONS

This application claims the benefit of U.S. Provisional Application No. 60/660,419, filed Mar. 10, 2005.

### FIELD OF THE INVENTION

The present invention relates generally to the field of osteoporosis, and more specifically to compositions and methods for the treatment and prevention of osteoporosis and/or inflammatory joint disease.

### BACKGROUND OF THE INVENTION

Osteoporosis is described in general terms as a reduction in bone density with retention of a normal chemical composition. More specifically, osteoporosis is a generalized, progressive diminution of bone density, i.e. bone mass per unit volume, causing skeletal weakness. Approximately 30 to 40% of the skeletal mass must be lost in order to reliably diagnose osteoporosis by radiology. Contemporary medicine distinguishes between primary and secondary osteoporosis (The Merck Manual of Diagnosis and Therapy, 17th ed., 1999). Primary osteoporosis includes juvenile osteoporosis, rare but occurring in children and young adults; Type I or postmenopausal osteoporosis, occurring in women between the ages of 50 and 75; and Type II or age-associated or senile osteoporosis, usually occurring and men and women older than 70 years. Primary osteoporosis is characterized by a predominant osteoclast activity and a disruption of the feedback mechanism between the serum calcium level and the parathyroid hormone (PTH) secretion. It occurs mainly uniformly throughout the whole skeleton. Secondary osteoporosis, accounting for less than 5% of all osteoporosis cases, results from chronic conditions that contribute significantly to accelerated bone loss. These conditions include endogenous and exogenous thyroxine excess, hyperparathyroidism, malignancies, gastrointestinal diseases, medications, renal failure and connective tissue diseases. It starts mostly at the main skeleton and progresses centrifugally. Osteoporosis is characterized by pain in the respective bones, diffuse back pain, vertebral body collapse, pathological fractures, in particular, fracture of the neck of the femur. The goal of the management of all types of osteoporosis is therefore to decrease pain, to prevent fractures and to maintain body functions.

Osteoporosis is a common clinical feature and common complication in patients affected with chronic inflammatory diseases with joint manifestations. These include rheumatoid arthritis (RA), Juvenile Rheumatoid Arthritis (JRA), psoriatic arthritis, Reiter's syndrome (reactive arthritis), Crohn's disease, ulcerative colitis, and sarcoidosis (Orcel, P.; Cohen-Solal, M.; de Vernejoul, M. C., and Kuntz, D. [Bone demineralization and cytokines]. Rev Rhum Mal Osteoartic. September 1992; 59(6 Pt 2):16S-22S; Brown, J. H. and Deluca, S. A. The radiology of rheumatoid arthritis. Am Fam Physician. October 1995; 52(5):1372-80; De Vos, M.; De Keyser, F.; Mielants, H.; Cuvelier, C., and Veys, E. Review article: bone and joint diseases in inflammatory bowel disease. Aliment Pharmacol Ther. 1998 May; 12(5):397-404; Falcini, F.; Trapani, S.; Civinini, R.; Capone, A.; Ermini, M., and Bartolozzi, G. The primary role of steroids on the osteoporosis in juvenile rheumatoid patients evaluated by dual energy X-ray absorptiometry. J Endocrinol Invest. March 1996; 19(3):165-9; Scutellari, P. N. and Orzincolo, C. Rheumatoid arthritis: sequences. Eur J Radiol. May 1998; 27 Suppl 1:S31-8).

Rheumatoid arthritis is associated with a decrease in bone mass (Cortet, B.; Flipo, R. M.; Blanckaert, F.; Duquesnoy, B.; Marchandise, X., and Delcambre, B. Evaluation of bone mineral density in patients with rheumatoid arthritis. Influence of disease activity and glucocorticoid therapy. Rev Rhum Engl Ed. July 1997-Sep. 30, 1997; 64(7-9):451-8). Typical changes of an inflammatory arthritis include juxta-articular osteoporosis, cartilage loss, and cortical or marginal bone erosions (Lawson, J. P. and Steere, A. C. Lyme arthritis: radiologic findings. Radiology. January 1985; 154(1):37-43; Grassi, W.; De Angelis, R.; Lamanna, G., and Cervini, C. The clinical features of rheumatoid arthritis. Eur J Radiol. May 1998; 27 Suppl 1:S18-24).

Joint inflammation exerts both local and systemic effects on skeletal tissues. Three forms of bone disease (bone loss) have been described in rheumatoid arthritis, namely: focal bone loss affecting the immediate subchondral bone and bone at the joint margins; periarticular osteopenia adjacent to inflamed joints; and generalized osteoporosis involving the axial and appendicular skeleton (Goldring, S. R. and Gravallese, E. M. Mechanisms of bone loss in inflammatory arthritis: diagnosis and therapeutic implications. Arthritis Res. 2000; 2(1):33-7). During chronic inflammatory joint diseases, such as rheumatoid arthritis, synovial cells produce large amounts of cytokines leading to increased local bone resorption and juxta-articular bone destructions (Orcel, P.; Cohen-Solal, M.; de Vernejoul, M. C., and Kuntz, D. [Bone demineralization and cytokines]. Rev Rhum Mal Osteoartic. September 1992; 59(6 Pt 2):16S-22S).

Homocysteinemia (the accumulation of homocysteine in plasma and tissue) is the result of deficiencies of certain enzymes and/or substrates involved in the transmethylation pathways. It is caused by the accumulation of homocysteine and its two disulfides in plasma and tissue (Mudd et al., The Metabolic Basis of Inherited Disease, New York, McGraw-Hill, 1978, p. 458). Homocysteinemia is associated with juvenile arteriosclerosis, recurrent arterial and venous thromboembolic manifestations and osteoporosis. The latter may be due to the fact that homocysteine also interferes with collagen synthesis, and it is this interaction that may be significant in the development of defective bone matrix and osteoporosis (Am J Med Sci, 273, 1977, p. 120). In addition, studies have concluded that an increased homocysteine level appears to be a strong and independent risk factor for osteoporotic fractures (van Meurs et al. N Engl J Med. May 13, 2004; 350(20):2033-41; Cashman, K D. Nutr Rev. January 2005; 63(1):29-36). Folic acid has been described as a successful tool for the treatment of hyperhomocysteinemia (Brattstrom et al., Metabolism, Vol. 34, No. 11, 1985, p. 1073). For this reason, folic acid has been included in compositions to treat osteoporosis, for example, as described in U.S. Pat. Nos. 6,790,462, 6,881,419, and 4,902,718.

There is a drawback to these compositions because the biologically active form of folic acid, 5-methyltetrahydrofolate (5-MTHF), may not be fully available due to a common genetic mutation. For example, a study published in 2000 by Botto and Yang of the Centers for Disease Control and Prevention, in the American Journal of Epidemiology (Botto, L. D., and Yang, Q. American Journal of Epidemiology, Vol 151, Issue 9: 862-877) demonstrated that one in eight women have a genetic trait that can prevent proper metabolism of folic acid. The trait is classified as homozygosity for the T allele of the C677T polymorphism of the gene encoding the folate dependent enzyme 5,10-methylenetetrahydrofolate reductase (MTHFR). It was reported by Botto and Yang that the homozygous genotype can be present in more than 40 percent of Hispanic women. The homozygous genotype was also observed in other ethnic subgroups. In a study by Peng, F., Labelle, L. A., Rainey, B. J., Tsongalis, G. J. Int J Mol Med. 2001; 8: 509-511, the prevalence of a C677T or A1298C single nucleotide polymorphism (SNP) was investigated. Homozygosity for the C677T MTHFR SNP was detected in 16% and 10% of Caucasians and Hispanics, respectively. The frequency of the C677T heterozygous SNP for Caucasians and Hispanics was 56% and 52%, respectively. Because of the inability of some individuals to properly metabolize folic acid, there is a need for improved folic-acid containing compositions for the treatment of osteoporosis.

It is therefore an object of the present invention to provide improved compositions for the treatment and/or the prevention of osteoporosis and/or inflammatory joint diseases. It is a more specific object of the present invention to provide improved compositions containing folic acid.

It is another object of the invention to provide improved compositions for the treatment and/or the prevention of osteoporosis and/or inflammatory joint diseases in subjects with a folic acid metabolism deficiency.

### BRIEF SUMMARY OF THE INVENTION

Compositions and methods for the treatment of osteoporosis and/or inflammatory joint disease are provided herein. The compositions contain a folate, such as a reduced folate, and folic acid. The folate is preferably 5-methyltetrahydrofolate, and most preferably 5-methyl-(6S)-tetrahydrofolic acid. The folate and folic acid can be given in the same dosage unit or separate dosage units, and more than one dosage unit can be given per dose. The compositions may also contain one or more vitamins and minerals selected from vitamin B12, vitamin B6, vitamin D3, calcium, magnesium, and polyunsaturated fatty acids (PUFAs). These ingredients are optional, but preferable (especially the vitamins and minerals). The compositions may further contain one or more additional ingredients such as vitamins, minerals, and laxatives.

The compositions are useful in the treatment of all forms of osteoporosis, including primary osteoporosis and secondary osteoporosis, and/or inflammatory joint diseases, especially in patients having a folic acid metabolism deficiency. The compositions are particularly useful in the treatment of inflammatory joint diseases, with complications that include bone loss, fracture, and osteoporosis. In addition, the compositions are beneficial for the prevention of osteoporosis in subjects who do not yet have the disease, but who are at risk for getting osteoporosis, such as post-menopausal women, subjects with osteopenia (mid thinning of the bone mass), subjects with an inflammatory joint disease, or people who are over the age of 70.

### DETAILED DESCRIPTION OF THE INVENTION

I. Compositions

The compositions described herein are compositions containing therapeutically effective amounts of a folate, such as a reduced folate, and folic acid. The compositions may also contain an effective amount of one or more ingredients selected from vitamin B12, vitamin B6, vitamin D3, calcium, magnesium, and polyunsaturated fatty acids (eg. omega 3 or omega 6 fatty acids). These ingredients are optional, but preferable (especially the vitamins and minerals). The compositions may also optionally contain an effective amount of one or more additional ingredients such as iron, zinc, copper, vitamin B1, vitamin B2, vitamin E, vitamin C, biotin, panthothenic acid, niacinamide, vitamin A, and emollient laxatives. The folate and folic acid may be contained in the same dosage unit or in separate dosage units, and more than one dosage unit can be given per dose. In addition, the optional ingredients may be contained in the same dosage unit as the folate and/or folic acid or in a separate dosage unit(s).

The phrase “an effective amount” or “a therapeutically effective amount” as used herein includes an amount sufficient for prevention, treatment, or amelioration of one or more of the symptoms of osteoporosis, osteopenia and/or an inflammatory joint disease, and includes an amount which results in the effect that one or more of the symptoms of these diseases are ameliorated or otherwise beneficially altered.

As used herein “composition(s)” and “formulation(s)” are used interchangeably and include preparations such as multivitamins (with or without minerals and other nutrients); breakfast foods such as prepared cereals, toaster pastries and breakfast bars; dietary supplements; animal feed (for example pet foods) and animal feed supplements. As used herein, the term “nutritional preparation(s)” is encompassed by the terms “composition(s)” and “formulation(s)” and refers more specifically to multivitamin preparations (with or without minerals and other nutrients) and/or dietary supplements.

The compositions are useful in the treatment of all forms of osteoporosis including primary osteoporosis (juvenile osteoporosis, postmenopausal (Type I) osteoporosis and age-associated or senile (Type II) osteoporosis) and secondary osteoporosis (osteoporosis caused by chronic conditions and diseases such as, metabolic disease, connective tissue disease, bone marrow disease, immobilization, and drug use). In addition, the compositions are beneficial for the prevention of osteoporosis in subjects who do not yet have the disease, but are at risk for getting osteoporosis, such as such as post-menopausal women, patients with osteopenia (mid thinning of the bone mass), or people who are over the age of 70. The compositions are also useful in the treatment of osteoporosis and/or inflammation in patients affected with inflammatory joint diseases such as rheumatoid arthritis (RA), Juvenile Rheumatoid Arthritis (JRA), psoriatic arthritis, Reiter's syndrome (reactive arthritis), Crohn's disease, ulcerative colitis, sarcoidosis.

Furthermore, the compositions are beneficial for the treatment or prevention of osteoporosis and/or inflammatory joint diseases in men and women with a folate deficiency or a folic acid metabolic disorder. “Defective folate metabolic pathway” or “deficient folic acid metabolic pathway” or “folic acid metabolism disorder” refers to a less than normal, lack of, inhibited, or restricted production of folic acid pathway metabolites. The terms also refer to less than normal, deficient or defective levels of folic acid metabolites in a human or other animal.

A. Folates

As used herein, the term “folates” includes the anionic form of folic acid, folate, and natural and unnatural isomers of reduced folate or a pharmaceutically compatible salt or combination thereof. In a preferred embodiment, the folate is a reduced folate. The term “reduced folate” is used herein to refer to both natural and unnatural isomers of reduced folate. Reduced folates and compositions containing these compounds are well-known and described in the art, for example, in U.S. Pat. Nos. 5,997,915; 6,011,040; 6,441,168; 5,350,851; and 6,921,754. Natural isomers of reduced folate suitable for use in the compositions include, for example, (6S)-tetrahydrofolic acid, 5-methyl-(6S)-tetrahydrofolic acid, 5-formyl-(6S)-tetrahydrofolic acid, 10-formyl-(6R)-tetrahydrofolic acid, 5,10-methylene-(6R)-tetrahydrofolic acid, 5,10-methenyl-(6R)-tetrahydrofolic acid, and 5-formimino-(6S)-tetrahydrofolic acid. Other natural isomers of reduced folate include the polyglutamyl, such as the diglutamyl, triglutamyl, tetraglutamyl, pentaglutamyl, and hexaglutamyl, derivatives of (6S)-tetrahydrofolic acid, 5-methyl-(6S)-tetrahydrofolic acid, 5-formyl-(6S)-tetrahydrofolic acid, 10-formyl-(6R)-tetrahydrofolic acid, 5,10-methylene-(6R)-tetrahydrofolic acid, 5,10-methenyl-(6R)-tetrahydrofolic acid, and 5-formimino-(6S)-tetrahydrofolic acid.

Any or all of the natural isomers of reduced folate can be present in its chirally pure form, or, alternatively, the composition can optionally contain a molar amount of one or more unnatural isomers of reduced folate, such as (6R)-tetrahydrofolic acid, 5-methyl-(6R)-tetrahydrofolic acid, 5-formyl-(6S)-tetrahydrofolic acid, 10-formyl-(6S)-tetrahydrofolic acid, 5,10-methylene-(6S)-tetrahydrofolic acid, 5,10-methenyl-(6S)-tetrahydrofolic acid, 5-formimino-(6R)-tetrahydrofolic acid, and polyglutamyl derivatives thereof. The molar amount of the natural isomer of reduced folate can be equal to the molar amount of its corresponding unnatural isomer (as where the unnatural and natural isomer are present as a racemic mixture), or, preferably, the natural isomer of reduced folate can be present in a molar amount greater than the molar amount of the corresponding unnatural isomer. The total molar amount of the one or more natural isomers of reduced folate present in the composition can be between 5% and 200% of a human daily requirement for folate per a customarily consumed quantity of the composition. Natural isomers of reduced folates that are substantially chirally pure can be prepared by any suitable method, including, for example, by the method described in U.S. Pat. No. 5,350,851. Pharmaceutically compatible salts of the reduced folates may also be used in the compositions and should be both pharmacologically and pharmaceutically compatible salts such as, but not limited to, alkali or alkaline earth metal salts, preferably sodium, potassium, magnesium or calcium salts.

In a preferred embodiment, the reduced folate is 5-methyltetrahydrofolate. “5-methyltetrahydrofolate” is used herein to refer to the compound N-(5-methyl)-5,6,7,8-tetrahydropteroyl)-Lglutamic acid or a pharmaceutically acceptable salt thereof 1) as a racemate (5-methyl-(6R,S)-tetrahydrofolic acid), 2) in the form of the individual isomers, 5-methyl-(6R)-tetrahydrofolic acid and 5-methyl-(6S)-tetrahydrofolic acid, or 3) in a desired ratio of the individual isomers. 5-methyltetrahydrofolate can be used interchangeably with “5-methyl-tetrahydrofolic acid”, “5-methylTHF”, “L-methylfolate”, “L-methyltetrahydrofolate”. This compound is well-known and described in U.S. Pat. No. 5,997,915. Salt forms of this compound are also well-known and described in U.S. Pat. No. 6,441,168. In the most preferred embodiment, the 5-methyltetrahydrofolate is 5-methyl-(6S)-tetrahydrofolic acid.

Unlike folic acid, 5-methyltetrahydrofolate does not require enzymatic conversion to the biologically active compound. This enzymatic conversion process can be difficult for some individuals, especially those who carry a folate metabolic gene mutation. The population at risk, and the population that can benefit from the presence of 5-methyltetrahydrofolate supplementation, is much larger than previously believed. In addition, those individuals affected by a genetic mutation in the folate metabolic pathway, especially those mutations that affect 5-methyltetrahydrofolate production or function, can be aided through the administration of a composition comprising 5-methyltetrahydrofolate. Therefore, compositions comprising 5-methyltetrahydrofolate and other reduced folates, eliminate, reduce or lessen the consequences of 5-methyltetrahydrofolate genetic deficiencies associated with folate metabolism.

5-methyltetrahydrofolate has been shown to be an ingredient of high bioavailability. In this application “bioavailable” and “bioavailability” are interchangeable and refer to “the degree to which, or rate at which, a drug or other substance is absorbed or becomes available at the site of physiological activity after administration”. Preliminary research suggests that 5-methyltetrahydrofolate is as equally bioavailable as folic acid. In particular circumstances, host-related factors, such as gastrointestinal illness and pH of the small intestine, can influence the bioavailability of folic acid, because it can be best converted into the active form prior to transport across the blood-brain barrier. For the reasons described above, compositions and nutritional preparations containing reduced folate in combination with folic acid are more beneficial than those just containing folic acid.

Therapeutically effective amounts of folate (e.g. reduced folate) that may be used in the compositions and preparations described herein preferably ranges from about 400 μg to about 7 mg. In one embodiment, the amount of folate ranges from about 400 μg to about 4 mg. In a specific embodiment, the folate is 5-methyl-(6S)-tetrahydrofolic acid present in the compositions and preparations at a range of from about 0.5 mg to about 2 mg. In yet another embodiment, the folate (e.g. reduced folate) is given in a dose of from 400 μg to 7 mg, and preferably from 0.8 mg to 4 mg. In a specific embodiment, the folate is 5-methyl-(6S)-tetrahydrofolic acid given in a dose of from about 0.8 mg to about 2 mg.

B. Folic Acid

The compositions also comprise folic acid. Compositions comprising, for example, both the reduced folate, 5-methyltetrahydrofolate, and folic acid have the increased benefit of providing a readily available form of biologically active 5-methyltetrahydrofolate while simultaneously providing a longer term source of folate, folic acid. As discussed above, folic acid must undergo enzymatic conversion to the biologically active form. Therefore, the combination of 5-methyltetrahydrofolate and folic acid provides a longer term source of folates than the use of 5-methyltetrahydrofolate or another reduced folate alone, for example, as described in U.S. Pat. No. 6,812,215. Therapeutically effective amounts of folic acid that may be used in the compositions described herein preferably ranges from about 50 μg to about 7 mg. In another embodiment, the amount of folic acid present in the compositions described herein is from about 400 μg to about 6 mg, and preferably about 1 mg to about 5 mg. In a specific embodiment, the amount of folic acid present in the compositions and preparations described herein is about 1 mg to about 3 mg. In a further embodiment, the folic acid is given in a dose of from about 400 μg to about 6 mg, and preferably about 1 mg to about 5 mg. In a specific embodiment, folic acid is given in a dose of from about 3 mg to about 5 mg.

In one embodiment, the total amount of folate provided in the compositions can be represented as the sum of the folate (e.g. reduced folate) and the folic acid. In one embodiment, the total amount of folate present in the compositions ranges from about 0% to about 40% reduced folate, and about 60% to about 100% folic acid. In one embodiment, the total amount of folate in the compositions is about 400 μg to about 7 mg. In a preferred embodiment, the total amount of folate in the compositions and preparations is greater than 0.8 mg, preferably about 1 mg to about 5 mg, and more preferably about 1 mg to about 3 mg. In another embodiment, the total amount of folate is given in a dose greater than 0.8 mg, preferably from about 1 mg to about 7 mg, and preferably from about 4 mg to about 6 mg.

C. Preferred Ingredients

1. Calcium

The compositions optionally, but preferably include a calcium compound or derivatives thereof. Because most of the body's calcium is found in the bones, an adequate intake of calcium is essential to maximize and maintain bone density. A calcium-poor diet is a primary risk factor for osteoporosis. Calcium, combined with vitamin D, maintains or helps reduce the rate of bone loss that occurs with osteoporosis.

The amount of calcium in the compositions ranges from about 20 mg to about 2500 mg of calcium compound or derivative. In one embodiment, the amount of calcium in the compositions is in excess of 200 mg and ranges from about 250 mg to about 2000 mg. In a specific embodiment, the amount of calcium in the compositions ranges from about 500 mg to about 1000 mg. In another embodiment, calcium is given in a dose of from about 250 mg to about 2000 mg, and preferably from about 500 mg to about 1000 mg. Biologically-acceptable calcium compounds include, but are not limited to, any of the well known calcium supplements, such as calcium carbonate, calcium sulfate, calcium oxide, calcium hydroxide, calcium apatite, calcium citrate-malate, bone meal, oyster shell, calcium gluconate, calcium lactate, calcium phosphate, calcium levulinate, and the like. In a preferred embodiment, the calcium compound is calcium carbonate.

2. Vitamin B₁₂

The compositions may optionally include a vitamin B₁₂ or one of the three active forms: cyanocobalamin, hydroxocobalamin, or nitrocobalamin, or derivatives thereof. The derivatives of vitamin B₁₂ include compounds formed from vitamin B₁₂ that are structurally distinct from vitamin B₁₂, but that retain the active function of vitamin B₁₂. Non-limiting examples of such derivatives include methylcobalamin, deoxyadenosylobalamin, combinations thereof and the like. Because high levels of homocysteine are implicated in osteoporosis (as described above), B vitamins involved in homocysteine conversion, such as B₁₂, may be beneficial in reducing the risk of osteoporosis. The amount of vitamin B₁₂ in the instant compositions ranges from about 2 μg to about 700 μg and is preferably in excess of 12 μg. In one embodiment, the amount of vitamin B₁₂ in the compositions ranges from about 100 μg to about 600 μg. In a specific embodiment, the amount of vitamin B₁₂ in the compositions ranges from about 250 μg to about 500 μg. In another embodiment, vitamin B12 is given in a dose of from about 100 μg to about 600 μg, and preferably from about 400 μg to about 600 μg.

3. Vitamin D₃

The formulations may optionally contain vitamin D₃ (cholecalciferol) or derivatives thereof. Derivatives of vitamin D₃ include compounds formed from vitamin D₃ that are structurally distinct from vitamin D₃, but that retain the active function of vitamin D₃. The vitamin D₃ may be present in a single form or in various different forms in combination within the present compositions. Vitamin D is vital for calcium absorption in bones and to improve muscle strength. Adequate storage levels of vitamin D help keep bones strong and may help prevent osteoporosis in older adults, in non-ambulatory individuals (those who have difficulty walking and exercising), in post-menopausal women, and in individuals on chronic steroid therapy (LeBoff M S et al. J Am Med Assoc 1999;251:1505-11). The amount of vitamin D₃ in the compositions preferably ranges from about 1 IU to about 2000 IU. In one embodiment, the amount of vitamin D₃ in the compositions ranges from about 100 IU to about 1000 IU. In a specific embodiment, the amount of vitamin D₃ in the compositions ranges from about 200 IU to about 500 IU. In another embodiment, vitamin D3 is given in a dose of from about 200 IU to about 1000 IU, and preferably about 300 IU to about 500 IU.

4. Vitamin B₆

The formulations may optionally contain vitamin B₆ (pyridoxine) or derivatives thereof. Derivatives of vitamin B₆ include compounds formed from vitamin B₆ that are structurally distinct from vitamin B₆, but that retain the active function of vitamin B₆. The vitamin B₆ may be present in a single form or in various different forms in combination within the present compositions. Because high levels of homocysteine are implicated in osteoporosis (as described above), B vitamins involved in homocysteine conversion, such as B₆, may be beneficial in reducing the risk of osteoporosis. The amount of vitamin B₆ in the compositions preferably ranges from about 0.1 mg to about 200 mg. In one embodiment, the amount of vitamin B₆ in the compositions ranges from about 0.5 mg to about 50 mg. In a specific embodiment, the amount of vitamin B₆ in the compositions ranges from about 1 mg to about 5 mg. In another embodiment, vitamin B6 is given in a dose of from 0.5 mg to about 50 mg, preferably 0.5 to 10 mg, and most preferably about 2 mg to about 4 mg.

5. Magnesium

The compositions may optionally include a magnesium compound or derivatives thereof. Preferably, the amount of magnesium in the compositions ranges from about 5 mg to about 500 mg of magnesium compound or derivative, and is preferably in excess of 30 mg. In one embodiment, the amount of magnesium in the compositions ranges from about 10 mg to about 200 mg. In a specific embodiment, the amount of magnesium in the compositions ranges from about 20 mg to about 100 mg. In another embodiment, magnesium is given in a dose of from about 10 mg to about 500 mg, and more preferably from about 50 mg to about 150 mg. In a preferred embodiment, the magnesium compound is magnesium oxide. Biologically-acceptable magnesium compounds which may be incorporated into the present inventive subject matter include, but are not limited to, magnesium stearate, magnesium carbonate, magnesium oxide, magnesium hydroxide and magnesium sulfate.

6. Polyunsaturated Fatty Acids (PUFAs)

The formulations may optionally include a polyunsaturated fatty acid, such as omega-3, omega-6, or omega-9 fatty acids, or mixtures of polyunsaturated fatty acids. PUFAs are well-known and their use in the treatment of osteoporosis is described, for example, in U.S. Patent Application Publication Nos. 20040082523 and 20020198177. Alpha-linolenic (ALA), docosahexaenoic (DHA), and eicosapentaenoic (EPA) acids are examples of omega-3 fatty acids. Linoleic acid (LA) and arachidonic acid (AA) are examples of omega-6 fatty acids. Oleic (OA) and erucic acid (EA) are examples of omega-9 fatty acids. The Omega-3 and Omega-6 fatty acids are polyunsaturated fatty acids classified as essential because humans cannot synthesize fatty acids and must obtain them through the diet.

In a preferred embodiment, the PUFA is an omega-3 fatty acid, or a mixture of omega-3 fatty acids, and preferably contains docosahexaenoic acid (DHA). DHA and vitamin/mineral compositions containing this essential fatty acid are described in detail in U.S. Patent Publication No. 2003/0050341. DHA is one of the main components of brain and heart tissue. It is required for the proper functioning of all neural systems, including the brain, the retina and the central nervous system.

In one embodiment, the compositions may contain DHA that is substantially free (<10%, and preferably <5%) of other Omega-3 fatty acids. In another embodiment, the DHA may be relatively free (<10%, and preferably <5%) of Omega-6 fatty acids, such as linoleic acid. In one embodiment, the compositions may contain linoleic acid in concentrations less than or equal to 5% by weight of DHA raw materials. In another embodiment, the DHA component contains at least about 40% DHA relative to all other fatty acids. DHA is mainly available as a fish oil extract. However, compositions containing fish-derived DHA may have a potent, offensive taste or odor. Additionally, substances derived from fish are believed to contain contaminants such as pollutants or ocean-borne contaminants, including dioxin and mercury. Therefore, it is desirable to use DHA derived from a natural source, preferably a vegetarian or non-fish source, such as algae (e.g. Crypthecodinium cohnii). Methods for the production of DHA from algae are described in the following patents, U.S. Pat. Nos. 5,130,242, 5,340,742, 5,340,594, 6,451,567, 6,509,178, and 6,607,900.

The PUFA can be contained within the same dosage unit as the folate and folic acid, and optional other ingredients or may be in a separate dosage unit. In one embodiment, the PUFA may be provided as a separate capsule that is substantially free of other vitamins or minerals. For example, the compositions can contain at least one tablet containing a reduced folate and folic acid and at least one softgel EFA capsule presented together in one packing material. The PUFA may be presented in a hard capsule, such as, but not limited to, a hard gelatin capsule, or a soft gelatin (softgel) capsule. In one embodiment, a PUFA is presented in an encapsulated semi-solid or liquid form. In a another embodiment, the PUFA is DHA or mixture of polyunsaturated fatty acids containing DHA, which is presented in a semi-solid or liquid form packaged in a soft gelatin (softgel) capsule. In one embodiment, the soft gelatin capsule is prepared from vegetable or plant based materials. In a further embodiment, the soft gelatin capsules are made from cellulosic raw materials. The soft gelatin capsules may be preservative-free, easy to swallow, effectively mask taste and odor, and allow product visibility. Softgels may be prepared, for example, without limitation, by dispersing the formulation in an appropriate vehicle to form a high viscosity mixture. This mixture is then encapsulated with a gelatin or vegetable based material using technology and machinery known to those in the softgel industry.

Therapeutically effective amounts of PUFA that may be used in the compositions and preparations preferably range from about 100 mg to about 1 g. In one embodiment, the amount of the EFA present in the compositions and preparations ranges from about 200 mg to about 800 mg. In a specific embodiment, the essential fatty acid is DHA present in the compositions and preparations in a range of from about 250 mg to about 500 mg. In one embodiment, wherein the compositions comprise a DHA softgel capsule, the DHA softgel capsule may optionally contain DHA that is essentially free of other vitamins, minerals and Omega-3 fatty acids. In another embodiment, the DHA softgel capsule may optionally comprise a DHA softgel capsule that is essentially free of eicosapentaenoic acid and linolenic acid.

D. Additional Vitamins, Minerals, and Ingredients

1. Iron

The compositions may optionally include an iron compound or derivatives thereof. In one embodiment, the amount of iron in the compositions ranges from about 10 mg to about 200 mg of iron compound or derivative. In one embodiment, the iron compound is elemental iron. In a preferred embodiment, the iron compound is carbonyl iron in a range of from about 80 mg to about 130 mg, and preferably 90 mg. In an alternative embodiment, the iron compound is an iron salt or combinations thereof, including, but not limited to, ferrous sulfate, ferrous fumarate, ferrous succinate, ferrous gluconate, ferrous lactate, ferrous glutamate or ferrous glycinate in a range of from 20 mg to 80 mg.

2. Copper

The compositions may optionally include a copper compound or derivatives thereof. Preferably, the amount of copper in the compositions ranges from about 0.1 mg to about 10 mg of copper compound or derivative. In one embodiment, the amount of copper in the compositions ranges from about 1 mg to about 5 mg. In a specific embodiment, the amount of copper in the compositions ranges from about 1.5 mg to about 2.5 mg. In one embodiment, the copper compound is cupric oxide.

3. Zinc

The compositions may optionally include a zinc compound or derivatives thereof. Preferably, the amount of zinc in the compositions ranges from about 5 mg to about 100 mg of zinc compound or derivative. In one embodiment, the amount of zinc in the compositions ranges from about 10 mg to about 30 mg. In a specific embodiment, the amount of zinc in the compositions ranges from about 12 mg to about 20 mg. In a preferred embodiment, the zinc compound is zinc oxide.

4. Vitamin B₁

The formulations of the compositions described herein may optionally contain vitamin B₁ (thiamine mononitrate) or derivatives thereof. Derivatives of vitamin B₁ include compounds formed from vitamin B₁ that are structurally distinct from vitamin B₁, but that retain the active function of vitamin B₁. The vitamin B₁ may be present in a single form or in various different forms in combination within the present compositions. The amount of vitamin B₁ in the compositions preferably ranges from about 0.5 mg to about 50 mg. In one embodiment, the amount of vitamin B₁ in the compositions ranges from about 1 mg to about 4 mg. In a specific embodiment, the amount of vitamin B₁ in the compositions ranges from about 2 mg to about 3.5 mg.

5. Vitamin B₂

The formulations may optionally include vitamin B₂ (riboflavin) or derivatives thereof. Derivatives of vitamin B₂ include compounds formed from vitamin B₂ that are structurally distinct from vitamin B₂, but that retain the active function of vitamin B₂. The vitamin B₂ may be present in a single form or in various different forms in combination within the present compositions. The amount of vitamin B₂ in the compositions preferably ranges from about 0.5 μg to about 50 mg. In one embodiment, the amount of vitamin B₂ in the compositions ranges from about 1 mg to about 4.5 mg. In a specific embodiment, the amount of vitamin B₂ in the compositions ranges from about 3.0 mg to about 3.8 mg.

6. Vitamin E

The formulations may optionally include vitamin E (dl-alpha tocopheryl acetate) or derivatives thereof. Derivatives of vitamin E include compounds formed from vitamin E that are structurally distinct from vitamin E, but that retain the active function of vitamin E. The vitamin E may be present in a single form or in various different forms in combination within the present compositions. The amount of vitamin E in the compositions preferably ranges from about 1 IU to about 910 IU. In one embodiment, the amount of vitamin E in the compositions ranges from about 5 IU to about 500 IU. In a specific embodiment, the amount of vitamin E in the compositions ranges from about 8 IU to about 200 IU.

7. Vitamin C

The formulations described herein may optionally include vitamin C (ascorbic acid) or derivatives thereof. Derivatives of vitamin C include compounds formed from vitamin C that are structurally distinct from vitamin C, but that retain the active function of vitamin C. The vitamin C may be present in a single form or in various different forms in combination within the present compositions. The amount of vitamin C in the compositions preferably ranges from about 10 mg to about 2000 mg. In one embodiment, the amount of vitamin C in the compositions ranges from about 75 mg to about 1000 mg. In a specific embodiment, the amount of vitamin C in the compositions ranges from about 100 mg to about 500 mg.

8. Biotin

The formulations may optionally contain biotin or derivatives thereof. Derivatives of biotin include compounds formed from biotin that are structurally distinct from biotin, but that retain the active function of biotin. The biotin may be present in a single form or in various different forms in combination within the present compositions. The amount of biotin in the compositions preferably ranges from about 10 μg to about 50 μg. In one embodiment, the amount of biotin in the compositions ranges from about 20 μg to about 40 μg. In a specific embodiment, the amount of biotin in the compositions ranges from about 25 μg to about 35 μg.

9. Pantothenic Acid

The formulations may optionally include pantothenic acid (calcium pantothenate) or derivatives thereof. Derivatives of pantothenic acid include compounds formed from pantothenic acid that are structurally distinct from pantothenic acid, but that retain the active function of pantothenic acid. The pantothenic acid may be present in a single form or in various different forms in combination within the present compositions. The amount of pantothenic acid in the compositions preferably ranges from about 1 mg to about 10 mg. In one embodiment, the amount of pantothenic acid in the compositions ranges from about 3 mg to about 8 mg. In a specific embodiment, the amount of pantothenic acid in the compositions ranges from about 5 mg to about 7 mg.

10. Niacinamide

The formulations may optionally include niacinamide or derivatives thereof. Derivatives of niacinamide include compounds formed from niacinamide that are structurally distinct from niacinamide, but that retain the active function of niacinamide. The niacinamide may be present in a single form or in various different forms in combination within the present compositions. The amount of niacinamide in the compositions preferably ranges from about 1 mg to about 100 mg. In one embodiment, the amount of niacinamide in the compositions ranges from about 10 mg to about 30 mg. In a specific embodiment, the amount of niacinamide in the compositions ranges from about 15 mg to about 25 mg.

11. Vitamin A

The formulations may optionally include vitamin A from any commonly known source, for example, retinol or beta-carotene. Preferably, the source of vitamin A is beta-carotene. In one embodiment, vitamin A is provided in a total daily dose of between 0-10,000 I.U, and preferably between 2,000 and 5,000 IU.

12. Emollient Laxatives

In one embodiment, the compositions optionally include an emollient laxative. The term “emollient laxative” is used herein to define a stool softener. In one embodiment, the emollient laxative is sodium docusate, glycerin, mineral oil or a poloxamer. In another embodiment, the emollient laxative is a pharmaceutically acceptable salt of docusate, such as, but not limited to, calcium. In another embodiment, the amount of emollient laxative provided in the instant compositions is between approximately 50 mg and approximately 1 g. In another embodiment, the amount of emollient laxative in the composition is about 50 to about 200 mg.

D. Salts and Derivatives

Although described above with reference specific to compounds, one can also utilize enantiomers, stereoisomers, derivatives and salts of the active compounds. Methods for synthesis of these compounds are known to those skilled in the art. Examples of pharmaceutically acceptable salts include, but are not limited to, mineral or organic acid salts of basic residues such as amines, and alkali or organic salts of acidic residues such as carboxylic acids. The pharmaceutically acceptable salts include the conventional non-toxic salts or the quaternary ammonium salts of the parent compound formed, for example, from non-toxic inorganic or organic acids. Conventional non-toxic salts include those derived from inorganic acids such as hydrochloric, hydrobromic, sulfuric, sulfamic, phosphoric and nitric acid ; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, tolunesulfonic, methanesulfonic, ethane disulfonic, oxalic and isethionic acids. The pharmaceutically acceptable salts can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed. (Mack Publishing Company, Easton, Pa., 1985, p. 1418).

E. Formulations

The folate and folic acid may be contained within the same dosage unit or in separate dosage unit. In a preferred embodiment, they are contained in the same dosage unit. As used herein, “dosage unit” means any pharmaceutically acceptable form for administering a drug to a patient, including, but not limited to, capsules, tablets, buccal forms, troches, lozenges and oral liquids, suspensions or solutions. The preferred, but optional, ingredients can be given 1) in the same dosage unit as the folate and folic acid if they are formulated together, 2) in the same dosage unit as either the folate or folic acid if they are given in separate dosage units or 3) in a completely separate dosage unit from both the folic acid and the folate. In a preferred embodiment, the preferred ingredients are formulated in the same dosage unit as the folate and folic acid. In a preferred embodiment, the reduced folate and folic acid and optional ingredients are formulated into a tablet or capsule.

Film coated tablets, for example, without limitation, may be prepared by coating tablets using techniques such as, but not limited to, rotating pan coating methods or air suspension methods to deposit a contiguous film layer on a tablet. This procedure is often done to improve the aesthetic appearance of tablets, but may also be done to improve the ease of swallowing of tablets, or to mask an odor or taste. The compositions may conveniently be presented in unit dosage form and may be prepared by conventional pharmaceutical techniques. The compositions may be provided in a blister-pack or other such pharmaceutical package, without limitation Preferably, the compounds are orally administered. For oral administration, the compounds, particularly their acid addition salts, are formed into tablets, granules, powders or capsules containing suitable amounts of granules or powders by a conventional method together with usual drug additives. Oral formulations containing the active compounds may be in any conventionally used oral form, including tablets, capsules, buccal forms, troches, lozenges and oral liquids, suspensions or solutions. Oral formulations may utilize standard delay or time release formulations to alter the absorption of the active compound(s).

Formulation of drugs is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980). The active compounds (or pharmaceutically acceptable salts thereof) may be administered in the form of a pharmaceutical composition wherein the active compound(s) is in admixture or mixture with one or more pharmaceutically acceptable carriers, excipients or diluents. Pharmaceutical compositions may be formulated in conventional manner using one or more physiologically acceptable carriers comprising excipients and auxiliaries which facilitate processing of the active compounds into preparations which can be used pharmaceutically.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Additionally, the coating material may contain conventional carriers such as plasticizers, pigments, colorants, glidants, stabilization agents, pore formers and surfactants.

Optional pharmaceutically acceptable excipients present in the drug-containing tablets, capsules, beads, granules or particles include, but are not limited to, diluents, binders, lubricants, disintegrants, colorants, stabilizers, and surfactants. Diluents, also referred to as “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar.

Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

Lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone XL from GAF Chemical Corp).

Stabilizers are used to inhibit or retard drug decomposition reactions which include, by way of example, oxidative reactions.

Surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-.beta.-alanine, sodium N-lauryl-.beta.-iminodipropionate, myristoamphoacetate, lauryl betaine and lauryl sulfobetaine.

If desired, the tablets, beads, granules, or particles may also contain minor amount of nontoxic auxiliary substances such as wetting or emulsifying agents, dyes, pH buffering agents, or preservatives.

Blending or copolymerization sufficient to provide a certain amount of hydrophilic character can be useful to improve wettability of the materials. For example, about 5% to about 20% of monomers may be hydrophilic monomers. Hydrophilic polymers such as hydroxylpropylcellulose (HPC), hydroxpropylmethylcellulose (HPMC), carboxymethylcellulose (CMC) are commonly used for this purpose. Also suitable are hydrophobic polymers such as polyesters and polyimides. It is known to those skilled in the art that these polymers may be blended with polyanhydrides to achieve compositions with different drug release profiles and mechanical strengths. Preferably, the polymers are bioerodable, with preferred molecular weights ranging from 1000 to 15,000 kDa, and most preferably 2000 to 5000 Da.

The compounds may be complexed with other agents as part of their being pharmaceutically formulated. The pharmaceutical compositions may take the form of, for example, tablets or capsules prepared by conventional means with pharmaceutically acceptable excipients such as binding agents (e.g., acacia, methylcellulose, sodium carboxymethylcellulose, polyvinylpyrrolidone (Povidone), hydroxypropyl methylcellulose, sucrose, starch, and ethylcellulose); fillers (e.g., corn starch, gelatin, lactose, acacia, sucrose, microcrystalline cellulose, kaolin, mannitol, dicalcium phosphate, calcium carbonate, sodium chloride, or alginic acid); lubricants (e.g. magnesium stearates, stearic acid, silicone fluid, talc, waxes, oils, and colloidal silica); and disintegrators (e.g. micro-crystalline cellulose, corn starch, sodium starch glycolate and alginic acid. If water-soluble, such formulated complex then may be formulated in an appropriate buffer, for example, phosphate buffered saline or other physiologically compatible solutions. Alternatively, if the resulting complex has poor solubility in aqueous solvents, then it may be formulated with a non-ionic surfactant such as TWEEN™, or polyethylene glycol. Thus, the compounds and their physiologically acceptable solvates may be formulated for administration.

Delayed release and extended release compositions can be prepared according to methods readily known in the art. The delayed release/extended release pharmaceutical compositions can be obtained by complexing drug with a pharmaceutically acceptable ion-exchange resin and coating such complexes. The formulations are coated with a substance that will act as a barrier to control the diffusion of the drug from its core complex into the gastrointestinal fluids. Optionally, the formulation is coated with a film of a polymer which is insoluble in the acid environment of the stomach, and soluble in the basic environment of lower GI tract in order to obtain a final dosage form that releases less than 10% of the drug dose within the stomach

Examples of rate controlling polymers that may be used in the dosage form are hydroxypropylmethylcellulose (HPMC) with viscosities of either 5, 50, 100 or 4000 cps or blends of the different viscosities, ethylcellulose, methylmethacrylates, such as Eudragit RS100, Eudragit RL100, Eudragit NE 30D (supplied by Rohm America). Gastrosoluble polymers, such as Eudragit E100 or enteric polymers such as Eudragit L100-55D, L100 and S100 may be blended with rate controlling polymers to achieve pH dependent release kinetics. Other hydrophilic polymers such as alginate, polyethylene oxide, carboxymethylcellulose, and hydroxyethylcellulose may be used as rate controlling polymers.

II. Methods of Use

A. Disorders to be Treated

1. Osteoporosis

In one embodiment, the compositions may be used to treat patients with all forms of osteoporosis. Osteoporosis is a systemic skeletal disease characterized by low bone mass and microarchitectural deterioration of bone tissue, with a consequent increase in bone fragility. Consequently, many individuals, both male and female, experience pain, disability, and diminished quality of life (QOL) caused by osteoporosis. The World Health Organization (WHO) has established the following definitions of osteoporosis based on bone mass density measurements in white women: Normal—Bone density no lower than 1 standard deviation (SD) below the mean for young adult women (T-score above −1); Low bone mass (osteopenia)—Bone density 1.0-2.5 SD below the mean for young adult women (T-score between −1 and −2.5); Osteoporosis—Bone density 2.5 SD or more below the normal mean for young adult females (T-score at or below −2.5).

Osteoporosis has been divided into several classifications according to etiology and localization in the skeleton. Osteoporosis initially is divided into localized and generalized categories. These 2 main categories are classified further into primary and secondary osteoporosis.

Primary osteoporosis occurs in patients in whom a secondary cause of osteoporosis cannot be identified, including juvenile and idiopathic (type I and type II) osteoporosis. Juvenile osteoporosis usually occurs in children or young adults (approx 8-14 years old) of both sexes. These patients have normal gonadal function. The first sign of juvenile osteoporosis is usually pain in the lower back, hips, and feet, often accompanied by difficulty walking. There may also be knee and ankle pain and fractures of the lower extremities. Physical malformations also may be present. These include abnormal curvature of the upper spine (kyphosis), loss of height, a sunken chest, or a limp. These physical malformations are sometimes reversible after the juvenile osteoporosis has run its course. Type I osteoporosis (postmenopausal osteoporosis) usually occurs in women aged 50-65 years. This type of osteoporosis is characterized by a phase of accelerated bone loss, primarily from trabecular bone. In this phase, fractures of the distal forearm and vertebral bodies are common. Type II osteoporosis (age-associated or senile) occurs in both women and men older than 70 years. This form of osteoporosis represents bone loss associated with aging. Fractures comprise both cortical and trabecular bone. In addition to wrist and vertebral fractures, hip fractures often are seen in type II osteoporosis.

Secondary osteoporosis occurs when an underlying disease or chronic condition causes osteoporosis. This includes endogenous and exogenous thyroxine excess, hyperparathyroidism, malignancies, gastrointestinal diseases, medications, renal failure and connective tissue diseases, bone marrow disease, immobilization, and drug use. Even the clinical history may not be completely revealing, as a patient with known metastatic disease can develop compression fractures from osteoporosis secondary to chemotherapy or administration of steroids, and radiation therapy can weaken the bone.

In another embodiment, the compositions may be used to treat subjects who do not yet have osteoporosis, but who are at risk for getting osteoporosis, such as post-menopausal women, patients with osteopenia (mild thinning of the bone mass), subjects with chronic inflammatory joint diseases (described below) or people who are over the age of 70. Osteopenia results when the formation of bone (osteoid synthesis) is not enough to offset normal bone loss (bone lysis). Osteopenia is generally considered the first step along the road to osteoporosis. Diminished bone calcification, as seen on plain X-ray film, is referred to as osteopenia, whether or not osteoporosis is present. The diagnosis of osteopenia may also be made by a special X-ray machine for bone density testing. Other risk factors for osteoporosis, such as age and bone density, have been established by virtue of their direct and strong relationship to incidence of fractures; however, many other factors have been considered risk factors based on their relationship to bone density as a surrogate indicator of osteoporosis. Risk factors include the following: advanced age, female sex, white race, Asian ethnicity, family history of osteoporosis, small body frame, amenorrhea, late menarche, early menopause, null parity, physical inactivity, alcohol and tobacco use, androgen or estrogen deficiency, and calcium deficiency.

2. Inflammatory Joint Diseases

In another embodiment, the compositions may be used to treat patients affected with inflammatory joint diseases, with complications that include bone loss, fracture, and osteoporosis. For example, studies have found an increased risk of bone loss and fracture in individuals with rheumatoid arthritis and juvenile rheumatoid arthritis. People with these diseases are at increased risk for osteoporosis for many reasons: 1) glucocorticoid (corticosteroid) medications such as prednisone often prescribed for the treatment of rheumatoid arthritis or juvenile rheumatoid arthritis can trigger significant bone loss; 2) pain and loss of joint function caused by the diseases can result in inactivity, further increasing osteoporosis risk; 3) studies also show that bone loss in rheumatoid arthritis may occur as a direct result of the disease. The bone loss is most pronounced in areas immediately surrounding the affected joints. Other known diseases with similar complications that may be treated with the compositions include psoriatic arthritis, Reiter's syndrome (reactive arthritis), Crohn's disease, ulcerative colitis, and sarcoidosis. The compositions can be beneficial for 1) subjects who have one or more chronic inflammatory joint diseases along with bone loss, fracture or osteoporosis or 2) subjects who have one or more chronic inflammatory joint diseases and who do not have bone loss, fracture, or osteoporosis, but are at risk for these complications.

B. Administration Protocol

The compositions of the present invention may involve the administration of the compositions at one or more times during a 24 hour period. For example, the compositions may be administered as a single dose of one or more tablets or capsules during a 24 hour period of time. In a preferred embodiment, the compositions are administered in a once daily dose.

An intermittent administration protocol may be used where chronic administration is not desirable. The compound or formulation is administered in time blocks of several days with a defined minimum washout time between blocks. Intermittent administration occurs over a period of several weeks to months to years to achieve a significant improvement in the symptoms of osteoporosis and chronic joint inflammation.

The compounds can be administered for a specific duration to improve symptoms of osteoporosis and/or chronic joint inflammation. A suitable endpoint can be where one symptom of the disorder is treated by administration of the compound and the treatment considered effective. In other situations, the treatment can be considered effective when more than one symptom is treated. In still other situations, the compositions may need to be administered to the patient for the duration of his or her life to prevent or treat osteoporosis and/or inflammatory joint disease, such as those patients with senile osteoporosis. The compositions may be modified in dosage as required by one skilled in the art. In one embodiment, the dosage can be modified by one skilled in the art to treat or prevent a disease or disorder, or lessen the risks associated with osteoporosis and or joint inflammation.

### EXAMPLES

### Example 1

The following composition may be used for the treatment and or prevention of osteoporosis and/or inflammatory joint disease:

A tablet containing:

| | | |
|---|---|---|
| Calcium | 20-2500 | mg; |
| Reduced Folate | 400-7,000 | mcg; |
| Folic acid | 50-6,000 | mcg; |
| Vitamin D₃ (cholecalciferol) | 1-2,000 | IU; |
| Vitamin B₆ (pyridoxine) | 0.1-200 | mg; |
| Vitamin B₁₂ (cyanocobalamin) | 2-250 | mcg; |
| Magnesium | 5-400 | mg; |

### Example 2

A similar composition as that described in Example 1 with the preferred amounts of the vitamins, minerals, and ingredients is as follows. Metafolin® (5-methyl-(6S)-tetrahydrofolic acid, calcium salt) is commercially available from Merck Eprova AG (Schaffausen, Switzerland).

A tablet containing:

| | | |
|---|---|---|
| Calcium (calcium carbonate, USP) | 500 | mg; |
| Metafolin ® | 500 | mcg; |
| Folic acid, USP | 2 | mg; |
| Vitamin D₃ (cholecalciferol) | 200 | IU; |
| Vitamin B₆ (pyridoxine HCl) | 1.2 | mg; |
| Vitamin B₁₂ (cyanocobalamin) | 250 | mcg; |
| Magnesium (magnesium oxide, USP) | 50 | mg; |

Two tablets containing the above formulation are given as a single dose.

Compositions incorporating the above formulation are prepared using conventional methods and materials known in the pharmaceutical art. The resulting folate supplements are recovered and stored for future use.

It is understood that the disclosed methods are not limited to the particular methodology, protocols, and reagents described as these may vary. It is also to be understood that the terminology used herein is for the purpose of describing particular embodiments only, and is not intended to limit the scope of the present invention which will be limited only by the appended claims.

Unless defined otherwise, all technical and scientific terms used herein have the same meanings as commonly understood by one of skill in the art to which the disclosed invention belongs.

Those skilled in the art will recognize, or be able to ascertain using no more than routine experimentation, many equivalents to the specific embodiments of the invention described herein. Such equivalents are intended to be encompassed by the following claims.

We claim:

1. A composition for the treatment or prevention of osteoporosis or inflammatory joint disease comprising a reduced folate, vitamin B12, vitamin B6, vitamin D3, magnesium and calcium in excess of 200 mg.

2. The composition of claim 1, wherein the reduced folate is selected from the group consisting of 5-methyl-(6S)-tetrahydrofolic acid and 5-methyl-(6R,S)-tetrahydrofolic acid.

3. The composition of claim 2, wherein the composition comprises 0.5 mg of 5-methyl-(6S)-tetrahydrofolic acid, 250 micrograms of vitamin B12, 1.2 mg of vitamin B6, 200 IU of vitamin D3, 50 mg of magnesium, and 500 mg of calcium.

4. A method of treating osteoporosis in a patient in need thereof comprising administering a therapeutically effective amount of a reduced folate to the patient.

5. The method of claim 4 wherein the reduced folate is administered in one dosage unit.

6. The method of claim 5, wherein more than one unit is administered per dose.

7. The method of claim 4 further comprising administering a therapeutically effective amount of one or more compounds selected from the group consisting of vitamin B12, vitamin B6, vitamin D3, calcium, magnesium, folic acid, and a polyunsaturated fatty acid or mixture of polyunsaturated fatty acids.

8. The method of claim 4, wherein the reduced folate is selected from the group consisting of 5-methyl-(6S)-tetrahydrofolic acid and 5-methyl-(6R,S)-tetrahydrofolic acid.

9. The method of claim 4, wherein the patient has a folic acid metabolism deficiency.

10. The method of claim 4, wherein the patient is an inflammatory joint disease.

11. A method of preventing osteoporosis in a patient at risk of developing osteoporosis comprising administering a therapeutically effective amount of a reduced folate acid to the patient.

12. The method of claim 11, wherein the reduced folate is administered in one dosage unit.

13. The method of claim 12, wherein more than one unit is administered per dose.

14. The method of claim 11 further comprising administering a therapeutically effective amount of one or more compounds selected from the group consisting of vitamin B12, vitamin B6, vitamin D3, calcium, magnesium, folic acid and a polyunsaturated fatty acid or mixtures of polyunsaturated fatty acids.

15. The method of claim 11, wherein the reduced folate is selected from the group consisting of 5-methyl-(6S)-tetrahydrofolic acid and 5-methyl-(6R,S)-tetrahydrofolic acid.

16. The method of claim 11, wherein the patient has a folic acid metabolism deficiency.

17. The method of claim 11, wherein the patient is selected from the group consisting of a post-menopausal woman, a subject with osteopenia, a subject with chronic inflammatory joint disease, and a patient over the age of 70.

18. A method of treating a chronic inflammatory joint disease in a patient in need thereof comprising administering a therapeutically effective amount of a reduced folate to the patient.

19. The method of claim 18 further comprising administering a therapeutically effective amount of one or more compounds selected from the group consisting of vitamin B12, vita B6, vitamin D3, calcium, magnesium, folic acid, and a polyunsaturated fatty acid and or mixture of polyunsaturated fatty acids.

20. The method of claim 18, wherein the disease is selected from the group consisting of rheumatoid arthritis, juvenile rheumatoid arthritis, psoriatic arthritis, Reiter's syndrome, Crohn's disease, ulcerative colitis, and sarcoidosis.
